(19) United States
(12) Patent Application Publication
(10) Pub. No.: US 2012/0197208 A1
(43) Pub. Date: Aug. 2, 2012

(54) Cap for a Portable Medical Delivery Device and Such a Medical Delivery Device

(75) Inventors: Ulrich Bruggemann, Frankfurt am Main (DE); Andrew Tubb, Guildford Surrey (GB); Christopher Langley, Leamington Spa (GB); Daniel Tyce, Leamington Spa (GB)

(73) Assignee: SANOFI-AVENTIS DEUTSCHLAND GMBH, Frankfurt am Main (DE)

(21) Appl. No.: 13/388,646

(22) PCT Filed: Aug. 5, 2010
(86) PCT No.: PCT/EP2010/061421

§ 371 Date: Apr. 18, 2012

(30) Foreign Application Priority Data
Aug. 12, 2009 (EP) 09008851.9

(51) Int. Cl. A61M 5/32

(52) U.S. Cl. 604/192

(57) ABSTRACT

Cap for a portable medical delivery device having an axial direction defining a first end and a second end of the cap comprising an opening (14) for receiving a front part of a medical delivery device defined at the first end of the cap, a socket (11) for covering a needle mounting portion (21) of the medical delivery device, characterized in that the cap further comprises a first receptacle (12) being designed for storing at least one spare needle (32) in the interior of the cap, and a medical delivery device comprising such a cap.

The present invention relates to a cap for a portable medical delivery device and such a medical delivery device.

Particularly, the present invention relates to a cap for a portable medical delivery device, having an axial direction defining a first end and a second end of the cap comprising an opening for receiving at least a front part of a medical delivery device defined at the first end of the cap and a socket for covering a needle mounting portion of the medical delivery device.

Such devices are used for instance by patients suffering from the disease of diabetes. This disease necessitates the application of an ingredient, known as insulin, several times a day, especially before the patient enjoys his meal. Therefore the patient carries along with him a portable medical delivery device, also known as an insulin syringe.

These devices typically contain a housing in which a receptacle or a cartridge comprising the insulin is integrated, as well as a dosage unit for regulating the dosage of the ingredient injected at each time the syringe is used. On the housing a low prized exchangeable needle is mounted for injecting the insulin under the skin of the patient. Such needles are, however, only usable for a predetermined number of injections and then have to be exchanged, so that the patients have to carry spare needles with them. For carrying spare needles along additional cases attachable to the medical delivery device are known in the prior art.

The patent application US 2008/0108951 A1 shows a storage system for needles pen delivery device syringe and a hypodermic needle adapted for attachment to and use with a pen delivery device syringe. Such a system, however, has the problem that the case for storing the spare needle is an additional part of the medical delivery device and must be separately mounted on the delivery device. Therefore on the one hand the number of parts during the production process of the medical delivery device is increased and on the other hand there remains the danger that the patient may forget or even lose the additional part.

From EP 0 927 056 B1 a syringe comprising a cap being located beside the needle mounted on the housing ready for use, the cap including a compartment in which at least one needle can be stored. With the provision of such a cap an additional part for providing a compartment for storing a needle is necessary which is uncomfortable for a user. Further, this solution is ergonomically disadvantageous as there might occur confusion for a patient in certain state of health as to which cap might be the right one to be used for injecting the medicament of the syringe.

It an object of the invention to provide a portable medical delivery device by which the use of the syringe is ergonomically helpful to a user with regard to the use of the needle of the syringe which must be removed from the syringe after an injection or which must be mounted before a next injection. A further object of the invention is to provide corresponding parts of the syringe in order to provide means for a better handling of a spare needle in an ergonomic way.

These objects are solved by a cap providing the feature of the independent claims. Further embodiments and examples of the invention are shown in the dependent claims which are referred back thereto.

According to an aspect of the invention, a cap for a portable medical delivery device is provided, comprising a needle for dispensing a pharmaceutical formulation therethrough in a distal direction, a front part and a needle mounting portion for mounting the needle, the cap having an axial direction defining a first end and a second end and comprising:
an opening at the first end of the cap for receiving the front part of the medical delivery device,
a socket for receiving the needle mounting portion of the medical delivery device.

In the interior of the cap at least one receptacle is formed which is designed for storing at least one needle in the interior of the cap, the receptacle having a bottom lying at the second end of the cap and an opening lying a distance extending from the bottom towards the first end of the cap.

With such an embodiment the spare needles can be stored directly in the cap of the portable medical delivery device and therefore be always attached to the device itself, such that no additional storing members are needed.

At least one receptacle of the cap can be designed for storing at least one spare needle.

According to this embodiment the sterile, unused, spare needles can easily be locked in a first receptacle, such that when the patient takes off the cap no sterile needle may fall out hazardously and therefore be damaged or get lost.

According to an example of the invention, the cap can comprise an opening and closing device which is disposed at the opening of the first receptacle, wherein the closing device comprises a cover which is moveable between a first position in which the opening is closed and a second position in which the opening is opened and vice versa, wherein the closing device is designed such that in its closed position a spare needle is kept inside the first receptacle and in its opened position a spare needle can be inserted in the first receptacle or can be removed from the first receptacle.

In a further embodiment of the present invention the cap may further comprise a second receptacle being designed for storing at least one used needle. In particular, in the interior of the cap comprises a receptacle being designed for storing at least one used needle, wherein the receptacle comprises a covering device disposed at an opening of the second receptacle and an opening and closing device which is moveable between a first position in which the opening is at least partly covered and a second position in which the opening is opened. In this example, the socket can be disposed between a first and a second receptacle with regard to the axial direction of the cap.

Generally, the covering device of the second receptacle being designed for storing at least one used needle can be realized such that a used needle can be introduced into the second receptacle but not removed from the second receptacle via the covering device by a user. In particular, the covering device can comprise a cover which is designed such that a used needle can be pressed through by a user such that the removal of the used needle through the same cover by the user is practically not possible. By this measure, the user will not take a used needle by mistake. In this regard, the covering device can be made of an elastic material and comprises an opening over which an elastic cover extends and which comprises a slot through which a used needle can be pressed. Further, the covering device can be designed such that the covering device or the cover can be removed from the opening. In this regard, the cover can be designed as flexible cover so that it can easily be mounted for example on an upper part or elevated edge of the receptacle or removed therefrom.

According to a further example of the invention, the receptacle comprising the covering device has a first end disposed at the first end of the cap and a second end disposed at a distance in the axial direction towards the first end of the cap, wherein the covering device is disposed at the first end of the cap.

According to an alternative example of the invention, the receptacle comprising the covering device has a first end disposed at the first end of the cap and a second end disposed at a distance in the axial direction towards the first end of the cap, wherein the covering device is disposed at the second end of the cap.

According to an alternative example of the invention, the receptacle comprising the covering device has a first end disposed at the first end of the cap and a second end disposed at a distance in the axial direction towards the first end of the cap, wherein the covering device is disposed at a side opening which extends from the inside of the portion of the receptacle comprising the covering device and a side wall of the cap.

According to a further example of the invention, the receptacle comprising the covering device has a first end disposed at the first end of the cap and a second end disposed at a distance in the axial direction towards the first end of the cap, wherein the receptacle comprising the covering device comprises an opening and opening and closing device disposed at the opening by which the covering device is moveable between a first position in which the opening is at least partly covered and a second position in which the opening is opened.

In this aspect of the invention, the opening at which the opening and closing device is disposed can be located at the first end of the receptacle.

Alternatively, the opening at which the opening and closing device is disposed can be located at the second end of the receptacle and wherein the opening and closing device comprises the covering device and wherein the opening and closing device is designed such that the covering device can be moved between an opened and a closed position.

As a further alternative, the opening at which the opening and closing device is disposed is lying at a side opening of the cap which extends from the inside of the portion of the receptacle comprising the covering device and a side wall of the cap.

With regard to the opening and closing device, the cap can comprise a locking device which is moveable between a locking state for locking the opening and closing device and a releasing state for opening the opening and closing device.

According to an example of the invention, the locking device comprises an operating element which is located at least partially outside the cap.

Generally, the locking device can be designed as a flexible element. Alternatively, the locking device can be designed as a latching device.

According to a further aspect of the invention, the locking device and/or the first receptacle being designed for storing at least one used needle, if any, and/or the second receptacle comprising the covering device, if any, is marked by a colour. In particular, the two receptacles of the cap can comprise coloured surfaces, wherein at least one surface of a first receptacle has a different colour than at least one surface of the other receptacle.

By using colours according to the invention, the user can securely and quickly distinguish visually the receptacle providing spare needles from the receptacle providing used needles and therefore confusion is prevented.

According to a further aspect of the invention, a medical delivery device can be provided, comprising a housing having an needle mounting portion for mounting a needle on the housing and a receptacle for storing a medicament to be delivered via the needle when being mounted on the needle mounting portion, wherein the needle mounting portion can be covered by a cap according to one embodiment or example of the invention. In particular, the needle mounting portion can be designed such that the needle can be detachably mounted on the needle mounting portion.

The above mentioned features may be combined partly or as a whole.

The term “medical delivery device” or “drug delivery device” according to instant invention shall mean a single-dose or multi-dose or pre-set dose or pre-defined dose, disposable or re-useable device designed to dispense a user selectable or pre-defined dose of a medicinal product, preferably multiple pre-defined doses, e.g. insulin, growth hormones, low molecular weight heparins, and their analogues and/or derivatives etc. Said device may be of any shape, e.g. compact or pen-type. The medication delivery device can be a pen-type device or an injector-type device. Furthermore, the said device may comprise a needle or may be needle-free. In addition, the said device may comprise a fixed needle or a replaceable needle or a moving needle or a shielded moving needle. In particular, the term “medical delivery device” shall mean a disposable needle-based pen-type device providing multiple pre-defined doses having mechanical and manual dose delivery and dose selection mechanisms, which is designed for use by persons without formal medical training such as patients.

Preferably, the drug delivery device is of the injector-type.

The medical delivery device comprises a drive mechanism for dispensing a set of a dose of a medicament from a medication receptacle of the delivery device in a distal direction. Dose selection or dose setting for selection or for setting a dose of a liquid or non-liquid medicament in a receptacle may be provided through a dosing mechanism or dose setting means or a dose setting mechanism so that the medical delivery device can further comprise a dose selection mechanism or dose setting mechanism for selection or setting the dose of a liquid medicament in the receptacle to be dispensed by the drive mechanism. The dosing mechanism can partly or totally be realized with components or functions of the drive mechanism or can be realized as a separate mechanism.

The term “distal end” according to instant invention shall mean the end of the device or a component of the device which is closest to the dispensing end of the device. The term “proximal end” according to instant invention shall mean the end of the device or a component of the device which is furthest away from the dispensing end of the device. Accordingly, the “distal direction” is the direction which is directed from the proximal end to the distal end of the device or a component of the device and the “proximal direction” is the direction which is directed from the distal end to the proximal end of the device or a component of the device.

Dose delivery may be provided through a mechanical (optionally manual) drive mechanism or drive means or electrical drive mechanism or electro-mechanical mechanism or stored energy drive mechanism, such as a spring, etc.. The drive mechanism can in particular comprise a piston being movable in the medication receptacle for displacing the medicament in the receptacle. At the piston a piston rod may be attached

The term “housing” according to instant invention shall preferably mean any exterior housing (“main housing”, “body”, “shell”) or interior housing (“insert”, “inner body”) having one or more helical threads. The housing may be designed to enable the safe, correct, and comfortable handling of the drug delivery device or any of its mechanism. Usually, it is designed to house, fix, protect, guide, and/or engage with any of the inner components of the drug delivery device (e.g., the drive mechanism, cartridge, plunger, piston rod) by limiting the exposure to contaminants, such as liquid, dust, dirt etc. In general, the housing may be unitary or a multipart component of tubular or non-tubular shape. Usually, the exterior housing serves to house a cartridge, which may be replaceable or non-replaceable, from which a number of doses of a medicinal product may by dispensed.

The invention will be understood in greater details from the following description of the preferred embodiments thereof, which are given only by way of example and with reference to the accompanying drawing, in which:

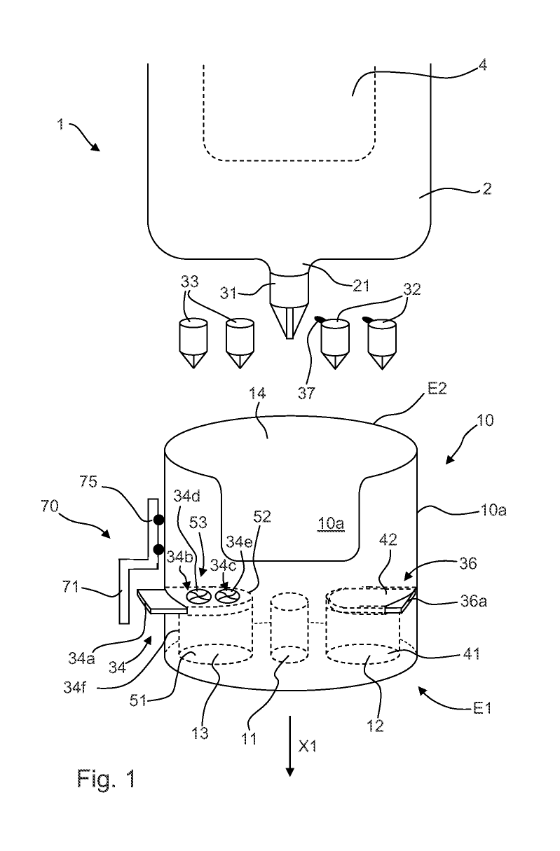

FIG. 1 is a perspective side view of a medical delivery device including an embodiment of a cap according to the present invention, the cap having a socket for receiving a needle mounting portion of the medical delivery device, one receptacle for storing at least one spare needle comprising a cover which is shown in a closed position and one receptacle for storing at least one used needle comprising a covering device which is disposed in the inner space of the cap,

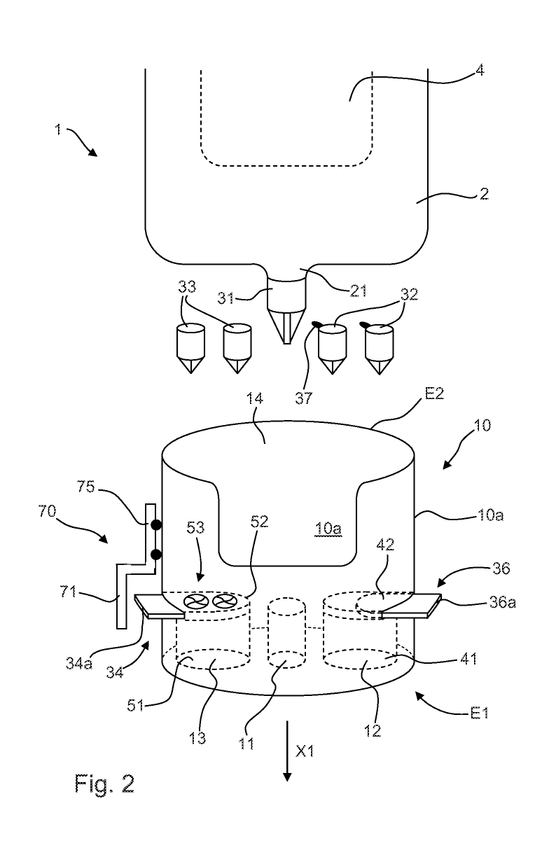

FIG. 2 is a perspective side view of the medical delivery device including the embodiment of a cap according to FIG. 1, wherein the cover of the receptacle for storing at least one spare needle is shown in an opened position,

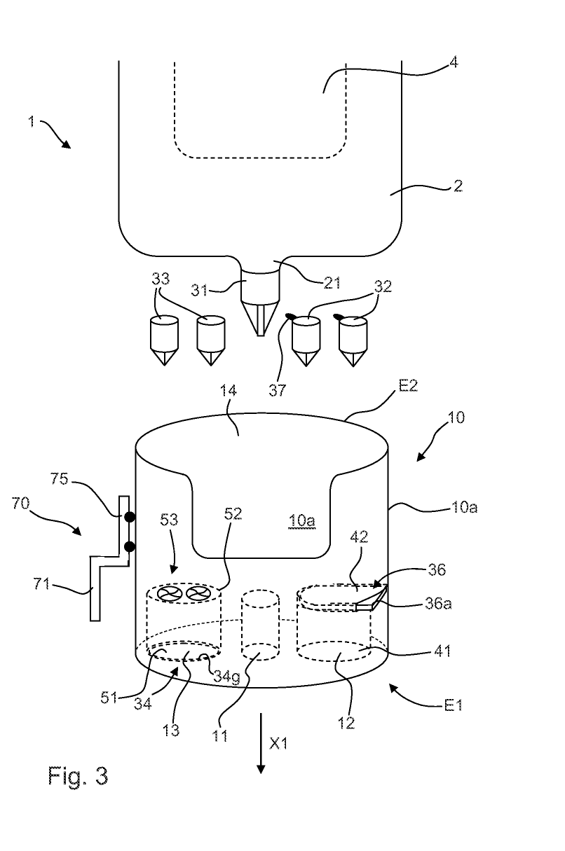

FIG. 3 is a perspective side view of the medical delivery device including the embodiment of a cap according to FIG. 1, wherein the cover of the receptacle for storing at least one used needle is disposed at the distal end of the cap.

Referring to the drawing in FIG. 1 a housing 2 is shown, which comprises a cartridge or receptacle 4 in which a medicament for injection is retained. The cartridge 4 is detachably mounted in the housing 2 and can particularly be adapted for dispensing a plurality of insulin dosages during a predetermined period like for instance one day. When activated the dosage is controlled by a driving mechanism and a dosing mechanism (not shown in the figures).

The housing 2 has a needle mounting portion 21 for receiving a needle 31. In the figures the needle 31 is provided with a storage cover which has to be taken away before injection.

In the figures, also spare needle assemblies 32 are shown in an embodiment which comprises a protecting cover having each a film 37 for closing the protecting cover. Before using the spare needle a patient has to take off the film 37 to take the new needle out off its cover.

Furthermore in FIG. 1 a cap 10 is shown which is of an essentially cylindrical shape having a first end or distal end E1 and a second end or proximal end E2 in its axial or longitudinal direction X1. At the first end of the cap an opening 14 is provided for receiving a front part of the medical delivery device 20 which comprises a needle mounting portion 21 is provided. At the second end the cap 10 is essentially closed. In an interior space or interior 10a of the cap 10 a socket 11 is provided for receiving the needle mounting portion 21 of the housing 2, wherein the socket 11 is designed for receiving the needle mounting portion 21 without needle mounted thereon and/or or a needle mounting portion 21 on which a needle 31 is mounted. The socket 11 can particularly be designed such that it may only receive the needle mounting portion 21, whereby the user is forced to remove a needle 31 used for an injection after each injection before mounting the cap 10 on the housing 2.

In the interior space 10a of the cap 10 a first receptacle 12 is disposed in which spare needles 32 or packages including spare needles may be introduced. The first receptacle has a first end or bottom 41 lying at a first or the distal end E1 of the cap 10 and an opening 43 lying at a second or proximal end 42 in a distance extending from the bottom 41 towards the second end E2 or the proximal end of the cap 10. At or on the opening 42 an opening and closing device 36 comprising a cover 36a is disposed which can be moved between an opened and a closed position. In FIG. 1 the cover 36a is shown in a closed position and in FIG. 2 the cover 36a is shown in an opened position. In an opened state of the first closing unit spare needles can be introduced or removed from the first receptacle 12. The opening and closing device 36 is operable by a user from the outside of the cap 10.

The first receptacle 12 may be designed such that spare needles 32 or packages including spare needles may only be contained in a predetermined orientation inside the receptacle 12. The opening 43 can be oriented towards the opening 14 (FIGS. 1 to 3) or can be disposed at the distal end E1 of the cap 10.

Alternatively or in addition, in the interior of the cap 10 a second receptacle 13 can be provided for receiving or for storing at least one used needle 33. The receptacle 12 can particularly comprise a covering device 34a which is disposed at an opening 53 of the second receptacle 12 and an opening and closing device 34 by which the covering device 34a is moveable between a first position in which the opening 53 is at least partly covered and a second position in which the opening 53 is opened. The opening 53 is located at the end of the receptacle 13 which is disposed at a distance from the distal end E1 towards the proximal end E2 of the cap 10.

The covering device 34a is designed such that a used needle 33 can be introduced into the second receptacle 13 but not removed from the second receptacle via the covering device 50 by a user. The covering device 34a according to the example shown in FIG. 1 comprises two openings 34b, 34c each of which are covered by a cover 34d, 34e which both are designed such that a used needle 33 can be pressed through by a user and such that the needle 33 which is inserted in the receptacle 13 can practically not be removed therefrom by the user.

In the embodiment shown in the figures, the covers 34d, 34e have a circular shape with at least two flaps and slots radially extending between the flaps. The flaps fold into the interior of the second receptacle 13 when a user pushes a used needle 33 through the covers 34d, 34e. However, the flaps are made of elastic material and designed such that they cannot be folded to the opposite side, being an outside of the second receptacle 13, so that it is not possible to pull a used needle out of the second receptacle 13 through the second closing unit 34, once it has been pushed inside. By moving the covering device 34a together with the covers 34d, 34e in its opened position, the receptacle is opened for removal of the needles from the receptacle 13. Such a covering device 34a with at least one cover 34d, 34e can alternatively be disposed at an opening which is disposed at the distal end 51 or at a side wall 34f of the receptacle.

Alternatively, the covering device 34a with at least one combination of an opening and a cover extending over the same can be fixedly disposed on the proximal end of the receptacle 13. In this embodiment, a further cover 34g can be disposed on the distal end or a side wall 34f of the receptacle 13 (FIG. 3).

In the embodiment shown in FIGS. 1 and 2, the socket 11 is disposed between the first and the second receptacle 12, 13 with regard to the axial direction X1 of the cap 10.

The opening and closing device 34 can be locked by a locking device 70 or latching device by means of an operating element 71, can be operated by a user. One embodiment of the locking device 70 is shown in the FIGS. 1 to 3 in a locking state.

For discharging the second receptacle 13 the user has to pull out the locking device 70 in a direction which is directed away from the cap 10 such that the covering device 34a can be moved in an opened position so that the used needles can be removed from the second receptacle 13 for example by the gravity force. The locking device 70 is provided at one side portion of the cap 10. For safety reasons, before pulling out the opening and closing device 34 the user has to move the locking unit 35 into an unlocked position.

Alternatively, the operating element 71 can be disposed by a guiding device 75 at a side wall of the cap by which the operating element 71 can be moved between an opened and a closed position by a linear movement.

For example, the receptacles 12, 13 and/or the opening and closing device 34 and/or the covering device 34a and/or the cover 36a can be marked by colours which in particular can differ from each other. Thereby, the user can easily identify the type of receptacle. Also, the opening and closing device 34 and the locking device 70 can be marked by the same colour

The above mentioned features or embodiments of the present invention may be combined partly or as a whole in any way.

1. Cap for a portable medical delivery device comprising a needle for dispensing a pharmaceutical formulation there through in a distal direction, a front part and a needle mounting portion for mounting the needle, the cap having an axial direction defining a distal end and a proximal end and comprising:
an opening at the first end of the cap for receiving the front part of the medical delivery device,
a socket for receiving the needle mounting portion of the medical delivery device,
characterized in that
in the interior of the cap at least one receptacle is formed which is designed for storing at least one needle in the interior of the cap, the receptacle having a bottom lying at the distal end of the cap and an opening lying a distance extending from the bottom towards the proximal end of the cap.

2. Cap according to claim 1, wherein an opening and closing device is disposed at the opening of the first receptacle, wherein the closing device comprises a cover which is moveable between a first position in which the opening is closed and a second position in which the opening is opened and vice versa, wherein the closing device is designed such that in its closed position a spare needle is kept inside the first receptacle and in its opened position a spare needle can be inserted in the first receptacle or can be removed from the first receptacle.

3. Cap according to claim 1, wherein in the interior of the cap comprises a receptacle being designed for storing at least one used needle, wherein the receptacle comprises a covering device disposed at an opening of the second receptacle and an opening and closing device which is moveable between a first position in which the opening is at least partly covered and a second position in which the opening is opened.

4. Cap according to claim 2, wherein the covering device of the second receptacle is designed such that a used needle can be introduced into the second receptacle but not removed from the second receptacle via the covering device by a user.

5. Cap according to claim 4, wherein the second receptacle comprises an opening over which a cover extends which is designed such that a used needle can be pressed through by a user.

6. Cap according to claim 5, wherein the covering device is made of an elastic material and comprises an opening over which an elastic cover extends and which comprises a slot through which a used needle can be pressed.

7. Cap according to claim 4, wherein the receptacle comprising the covering device has a first end disposed at the first end of the cap and a second end disposed at a distance in the axial direction towards the first end of the cap, wherein the covering device is disposed at the first end of the cap.

8. Cap according to claim 4, wherein the receptacle comprising the covering device has a first end disposed at the first end of the cap and a second end disposed at a distance in the axial direction towards the first end of the cap, wherein the covering device is disposed at the second end of the cap.

9. Cap according to claim 4, wherein the receptacle comprising the covering device has a first end disposed at the first end of the cap and a second end disposed at a distance in the axial direction towards the first end of the cap, wherein the covering device is disposed at a side opening which extends from the inside of the portion of the receptacle comprising the covering device and a side wall of the cap.

10. Cap according to claim 4, wherein the receptacle comprising the covering device has a first end disposed at the first end of the cap and a second end disposed at a distance in the axial direction towards the first end of the cap, wherein the receptacle comprising the covering device comprises an opening and opening and closing device disposed at the opening by which the covering device is moveable between a first position in which the opening is at least partly covered and a second position in which the opening is opened.

11. Cap according to claim 10, wherein the opening at which the opening and closing device is disposed is lying at the first end of the receptacle.

12. Cap according to claim 11, wherein the opening at which the opening and closing device is disposed is lying at the second end of the receptacle and wherein the opening and closing device comprises the covering device and wherein the opening and closing device is designed such that the covering device can be moved between an opened and a closed position.

13. Cap according to claim 12, wherein the opening at which the opening and closing device is disposed is lying at a side opening of the cap which extends from the inside of the portion of the receptacle comprising the covering device and a side wall of the cap.

14. Cap according to claim 10, wherein the cap comprises a locking device which is moveable between a locking state for locking the opening and closing device and a releasing state for opening the opening and closing device.

15. Medical delivery device, comprising a housing having an needle mounting portion for mounting a needle on the housing and a receptacle for storing a medicament to be delivered via the needle when being mounted on the needle mounting portion, characterized in that the needle mounting portion can be covered by a cap according to one of the preceding claims.
